(19) United States
(12) Patent Application Publication
(10) Pub. No.: US 2021/0032411 A1
(43) Pub. Date: Feb. 4, 2021

(54) SYNTHETIC, MUCUS-LIKE HYDROGEL AND METHOD OF PREPARATION, AND SYSTEM AND METHOD FOR PERFORMING MICRORHEOLOGY ON HYDROGELS AND OTHER COMPLEX FLUIDS

(71) Applicant: UNIVERSITY OF MARYLAND, COLLEGE PARK, College Park, MD (US)

(72) Inventors: Gregg DUNCAN, Columbia, MD (US); Katherine Joyner, Washington, DC (US); Bongsub Daniel Song, College Park, MD (US); Robert Hawkins, Thurmont, MD (US)

(21) Appl. No.: 16/945,830

(22) Filed: Aug. 1, 2020

Related U.S. Application Data
(60) Provisional application No. 62/882,333, filed on Aug. 2, 2019.
(60) Provisional application No. 62/934,859, filed on Nov. 13, 2019.

(51) Int. Cl. C08H 1/00; C08K 5/37; C08J 3/075; C08J 3/24; G01N 21/64; G01N 11/02

(52) U.S. Cl. CPC C08H 1/00; C08K 5/37; C08J 3/075; B82Y 5/00; G01N 21/6428; G01N 11/02; C08J 3/24

(57) ABSTRACT

A synthetic hydrogel is described, including hydrated mucin glycoproteins cross-linked with multi-arm thiol functional cross-linker, which can be prepared to model viscoelastic and micro-rheological properties of natural mucus. Such synthetic hydrogel can be prepared from a wide variety of mucin raw materials. Also described is a method of microrheologically characterizing mucus, by dispersing in the mucus muco-inert particles (MIP), irradiating the mucus containing MIP with polarized light, and measuring fluorescence polarization (FP) resulting from rotational diffusion of the MIP in the mucus in response to such irradiating, as a microrheological characteristic of the mucus. This method can be carried out using a plate reader equipped with a spectrofluorometer and polarized filter set, and therefore can be readily carried out in clinical settings without the necessity of specialized microrheological equipment.

### CROSS REFERENCE TO RELATED APPLICATIONS

The benefit under 35 USC §119 of U.S. Provisional Patent Application 62/882,333 filed Aug. 2, 2019 in the names of Bongsub Daniel Song, Katherine Joyner, and Gregg Duncan for Synthetic, Mucus-Like Hydrogel and Method of Preparation, and the benefit under 35 USC § 119 of U.S. Provisional Patent Application 62/934,859 filed Nov. 13, 2019 in the names of Robert Hawkins and Gregg Duncan for System and Method for Performing Micro rheology with Complex Fluids in a Conventional Plate Reader Format, are hereby claimed. The disclosures of U.S. Provisional Patent Application 62/882,333 and U.S. Provisional Patent Application 62/934,859 are hereby incorporated herein by reference in their respective entireties, for all purposes.

### FIELD

The present disclosure relates to synthetic, mucus-like hydrogel and method of making same, as well as a system and method for performing microrheology on hydrogel and other complex fluids.

### DESCRIPTION OF THE RELATED ART

Mucus is a biological gel that coats and protects epithelial surfaces in tissues throughout the body.

Mucins are large, polymeric glycoproteins that are the primary contributor to the viscoelastic properties of mucus, in which the cysteine-rich domains of mucins facilitate assembly into a fibrous network structure through disulfide cross-linking. Reversible, non-covalent bonds mediated through hydrophobic interactions and hydrogen bonds also contribute to the highly dynamic and complex arrangement of mucins within the gel. As a result, the physical properties of mucus gels are also influenced by buffer conditions where pH can vary from acidic (e.g. pH 2 in the stomach) to more neutral (e.g. pH 7 in the lung) conditions. Their network architecture allows mucus to macroscopically behave as a viscoelastic gel, and microscopically to function as a physical barrier, entrapping harmful pathogens and particulates to prevent their reaching the underlying epithelium.

The process by which mucins organize into a mucus gel has been difficult to replicate in the laboratory, and understanding of mucus properties has been limited by the lack of suitable models, which in turn has impeded the effort to develop engineered mucin-based materials that possess the natural anti-microbial properties of mucus, for biomedical applications.

In prior work, it has been observed that commercially available mucins, e.g., porcine gastric mucin, and bovine submaxillary mucin, do not form hydrogels at physiological pH or concentrations, presumably as a result of their processing. This considerably reduces the utility of such materials as models of natural mucus.

It has been shown that covalent cross-linking strategies utilizing acrylate- and glutaraldehyde-mediated bonds can be employed to form mucin gels, but these chemistries do not mirror the bond structure between mucin polymers in native mucus, which likewise limit their use as models in biological research. This has led to efforts to develop methods of purification of mucins without compromising their capacity to form gels, but such efforts have not succeeded in producing mucin-based materials that are generally useful as models of natural mucus.

Mucus secreted from tissue culture models has been collected and purified and shown to retain comparable viscoelastic behavior to human mucus, but the associated requirement of specialized, relatively low-yield processing techniques has limited the widespread usage of such models.

Accordingly, there is a need in the art for a model material that has the viscoelastic and rheological characteristics of natural mucus, and is readily producible in a simple and cost-effective manner.

In obstructive lung diseases such as asthma, cystic fibrosis (CF), and chronic obstructive pulmonary disease (COPD), mucus viscosity increases due to increased mucin production, increased DNA concentration caused by lysing of immune cells, and increased disulfide bonding. An inability to clear this highly viscoelastic mucus leads to reduced lung function, recurrent infections, and chronic inflammation.

Clinicians currently rely on indirect measures such as spirometry measurements (e.g., measurement of forced expiratory volume in 1 second, or FEV₁) and disease symptoms (e.g. cough, wheezing) to evaluate patients. However, these assessments fail to address how the viscoelastic properties of mucus produced in the lungs of these patients may impact disease progression. Accordingly, efforts have been made to develop techniques capable of directly detecting the physical changes of mucus associated with the diseased state.

Using particle tracking microrheology (PTM), the present inventors have previously shown that translational diffusion of polyethylene glycol (PEG) coated muco-inert nanoparticles (MIP) in mucus samples produced from patients with CF is correlated with disease severity (increased total solids content and viscoelasticity). This approach has shown a greater sensitivity to patient disease status than other leading biomarkers for obstructive lung diseases, and underscores the potential of utilizing microrheology for diagnosis, treatment, and prognosis of CF and other obstructive lung diseases. However, the technology required to perform PTM measurements is not available in clinical laboratories, since PTM is conventionally performed using microscopy and/or scattering techniques that require highly specialized equipment.

In the context of such incompatibility of PTM methodologies with standard clinical laboratory technologies, there is correspondingly a compelling need in the art for improved systems and methods for performing mucus microrheology measurements in a simple, direct, and cost-effective manner.

### SUMMARY

The present disclosure relates to synthetic, mucus-like hydrogel and method of preparation, as well as system and method for performing microrheology on hydrogels and other complex fluids.

In one aspect, the disclosure relates to a synthetic hydrogel, comprising hydrated mucin glycoproteins cross-linked with multi-arm thiol functional cross-linker.

In another aspect, the disclosure relates to a method of making a synthetic hydrogel, comprising: combining mucin in aqueous medium with a multi-arm thiol functional cross-linker; and cross-linking the mucin with the multi-arm thiol functional cross-linker to form the synthetic hydrogel.

In a further aspect, the disclosure relates to a method of microrheologically characterizing mucus, comprising:

dispersing in the mucus muco-inert particles (MIP);

irradiating the mucus containing MIP with polarized light; and

measuring fluorescence polarization (FP) resulting from rotational diffusion of the MIP in the mucus in response to said irradiating, as a microrheological characteristic of the mucus.

Other aspects, features and embodiments of the disclosure will be more fully apparent from the ensuing description and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a frequency sweep of 2% PGM (porcine gastric mucin) 2% 4arm-PEG-SH after 12 h, pH 7.4. At 1 rad/sec, G′≈175 pa and G″≈5.3 pa.

FIG. 2 shows the microrheology characteristics of 2% PGM/2% 4-arm PEG hydrogel, as determined by mean squared [MSD] displacement of 100-nm PEG coated nanoparticles after 12 h, and MSD after 30 minutes treatment of the hydrogel with 50 mM mucolytic N-acetylcysteine (NAC) at 37° C.

FIG. 3 schematically shows 100 nm and 500 nm polystyrene nanoparticles coated with polyethylene glycol (PEG), constituting muco-inert particles (MIP).

FIG. 4 shows the interaction of polarized light with MIP under low viscosity fast rotation conditions producing depolarized light with a low degree of polarization, and under high viscosity slow rotation conditions producing polarized light with a high degree of polarization.

FIG. 5 in panels A-D shows the microrheology results for mucin mixed with various thiol-based cross-linkers, in multiple particle tracking of 100-nm PEG-NP in mucin solutions with varying cross-linking polymers. Each panel represents the ensemble average mean squared displacement as function of time scale τ (MSD; <Δr2>) of 100 nm PEG-NP at hour 0 (triangles) and hour 9 (circles) in 2% w/v of PGM and 2% w/v of (A) PEG-1SH, (B) PEG-2SH, (C) PEG-4SH, and (D) dextran-SH. Reference lines with slope n=0.5 are included in each panel.

FIG. 6 shows the results for the microrheology of the 2% w/v PEG-4SH solution. Multiple particle tracking of 100-nm PEG-NP in 2% w/v PEG-4SH polymer solution produced the ensemble average mean squared displacement as a function of time scale τ (MSD; <Δr2>) of 100 nm PEG-NP at hour 0 (triangles) and hour 9 (circles). A reference line with slope n=0.5 is included.

FIG. 7 shows the results for microrheology of PEG-4SH mixed with hyaluronic acid. Multiple particle tracking of 100 nm PEG-NP in 2% w/v PEG-4SH mixed with 2% hyaluronic acid 500 kDa (Lifecore Biomedical) produced an ensemble average mean squared displacement as a function of time scale τ (MSD; <Δr²>) of 100 nm PEG-NP at hour 0 (triangles) and hour 9 (circles). A solid line with slope n=0.5 and a dashed line of 2% PGM/PEG-4SH hydrogel are also shown for reference.

FIG. 8 shows the effects of cysteine-blocking and disulfide cleavage on mucin-based hydrogel assembly and disassembly. Panel (A) shows MSD (<Δr2>) as a function of time scale (τ) of 100 nm PEG-NP in 2% w/v PGM and 2% w/v PEG-SH-4 solution 0 hours (downward triangles) and 9 hours (circles) after pre-treatment with iodoacetamide (IAM). Panel (B) shows MSD as a function of τ of 100 nm PEG-NP in 2% w/v PGM solution after 9 hours (PGM; upward triangles), in 2% PGM/PEG-4SH hydrogel after 9 hours (PGM Gel; circles), and in 2% PGM/PEG-4SH hydrogel after a 30-minute treatment with N-acetyl cysteine (PGM Gel+NAC; cross).

FIG. 9 shows the bulk rheological characterization of mucin-based hydrogels, for elastic modulus G′ (circles) and viscous modulus, G″ (squares) of PGM-based (panels A, B) and BSM-based (panels C, D) mucin hydrogels after a 24-hr gelation time. The data represent the average of 3 independently prepared hydrogels. Panel (A) shows strain (γ) sweep at frequency (ω) of 1 rad/s and panel (B) shows frequency sweep at γ=1% for 2% PGM and 2% PEG-4SH hydrogel. At ω=1 rad/s, the measured G′ was ˜244 Pa and tan δ was ˜8.1. Panel (C) shows strain (γ) sweep at frequency (ω) of 1 rad/s and panel (D) shows frequency sweep at γ=1% for 2% BSM and 2% PEG-4SH hydrogel. At ω=1 rad/s, G′ was measured as ˜270 Pa and tan δ was measured as ˜1.4.

FIG. 10 shows the impact of probe size on microrheology of mucin-based hydrogels, in panels A-F, for a 2% w/v PGM and 2% PEG-4SH gel measured using PEG-NP probes with diameters of 100 and 500 nm. Symbols in each panel are median values and bars indicate the interquartile range. Panels A and B show the MSD at τ=1 s (<Δr²(τ=1 s)>) for (A) 100 and (B) 500 nm PEG-NP during a 9-hour gelation period. Panels C and D show elastic to viscous moduli ratio (G′ /G″) at ω=1 rad/s calculated from measured MSD of (C) 100 nm and (D) 500 nm PEG-NP. Panels E and F show estimated pore size based on analysis of MSD at τ=1 s (ξ≈\sqrt{<Δr²(τ)>}+a; black circles) and G′ at ω=1 rad/s (k_{B}T/G′)^{1/3}; red squares) for (E) 100 nm and (F) 500 nm PEG-NP probes.

FIG. 11 shows gelation rate of mucin-based hydrogels with varying PGM and PEG-4SH concentration, in which kinetics of PGM/PEG-4SH gel formation were analyzed by microrheology using 100 nm PEG-NP probes, with gelation point measured as α=log₁₀[MSD]/log₁₀[τ]≤0.5, wherein each panel A and B displays the mean and standard error of measured α. Panel A shows a as a function of gelation time in hydrogels with a constant PEG-4SH concentration of 2% w/v and varying PGM concentrations of 1% w/v (black), 2% w/v (red), 3% w/v (green), 4% w/v (blue), and 5% w/v (brown). Panel B shows a as a function of gelation time in hydrogels with a constant PGM concentration of 2% w/v and varying PEG-4SH concentration of 1% w/v (black), 2% w/v (red), 3% w/v (green), 4% w/v (blue), and 5% w/v (brown).

FIG. 12 shows the gelation rate of mucin-based hydrogels with varying PGM concentration with a constant PEG-4SH concentration of 2% w/v, in which individual experiments used increasing PGM concentrations in w/v of: panel A, 1% (black); panel B, 2% (red); panel C, 3% (green); panel D, 4% (blue); and panel E, 5% (brown). Kinetics of PGM gel formation were analyzed by microrheology using 100 nm PEG-NP probes, with gelation point measured as α=0.5. Each of panels A-E displays the mean and standard error of measured a.

FIG. 13 shows the gelation rate of mucin-based hydrogels with varying PGM concentration with a constant PEG-4SH concentration of 2% w/v, in which individual experiments used increasing PEG-4SH concentrations in w/v of: panel A, 1% (black); panel B, 2% (red); panel C, 3% (green); panel D, 4% (blue); and panel E, 5% (brown). Kinetics of PGM gel formation were analyzed by microrheology using 100 nm PEG-NP probes, with gelation point measured as α=0.5. Each of panels A-E displays the mean and standard error of measured α.

FIG. 14 shows the decrease in MIP median (MSD) associated with increase in MIP polarization value in 1-5% PGM/2% PEG-4SH hydrogels for 100 nm MIP (graph A) and for 500 nm MIP (graph B).

FIG. 15 shows the increase in MIP polarization values with increasing mucin concentration in 1-5% PGM/2% PEG-4SH hydrogels for 100 nm MIP (graph A) and for 500 nm MIP (graph B).

FIG. 16 shows graphs for estimation of viscosity, with measured and reference data, using MIP rotational diffusion measurements in 60-100% water: glycerol mixtures for 100 nm MIP (graph A) and for 500 nm MIP (graph B).

FIG. 17 shows rotational diffusion results, for polarization values (Polarization (mP)) as a function of concentration, for rotational diffusion of 100 nm and 500 nm MIP in 1-5% porcine gastric mucin (PGM) with 2% 4-arm PEG-SH. Rotational diffusion of 100 nm and 500 nm MIP decreased with increasing mucin concentration. Polarization values were obtained through fluorescence polarization.

FIG. 18 shows rotational diffusion and log₀₁(MSD) of 100 nm and 500 nm MIP in 1-5% PGM with 2% 4-arm PEG-SH. Decrease in translational diffusion of 100 nm and 500 nm MIP was associated with decreased in corresponding rotational diffusion. MSD values were obtained through high-speed video microscopy and multiple particle tracking.

FIG. 19 shows expected (measured reference) viscosity values, and estimated viscosity values based on MIP rotational diffusion of 100 nm and 500 nm MIP, for viscosity of 0-100% w/w glycerol solutions. Accurate estimations of the viscosity of glycerol at various concentrations was obtained from both 100 nm and 500 nm MIP polarization values.

### DETAILED DESCRIPTION

The present disclosure relates to synthetic, mucus-like hydrogel and method of preparation thereof, as well as to system and method for performing microrheology on hydrogels and other complex fluids.

A synthetic hydrogel and method of preparation are described. Preparation techniques include utilization of thiol-based cross-linking to form mucin-based hydrogels that possess bulk rheological viscoelastic properties and network porosity that are comparable to those determined for native human mucus.

In one aspect, the disclosure relates to a synthetic hydrogel, comprising hydrated mucin glycoproteins cross-linked with multi-arm thiol functional cross-linker. In such synthetic hydrogel, the multi-arm thiol functional cross-linker may comprise thiol functionality at the termini of multiple ones of its arms. In various embodiments, the multi-arm thiol functional cross-linker has four arms, each of which is linked at a central organic core structure and extends outwardly therefrom, and comprises polyalkyloxy linear segments and a terminal thiol functionality. In various embodiments, each arm of the multi-armor thiol functional cross-linker comprises a chain structure of the formula —O(CH₂CH₂O)ₙCH₂CH₂SH wherein n is in a range of from 1 to 1000.

In the synthetic hydrogel of the present disclosure, the mucin glycoproteins may be of any suitable type, and may for example be porcine mucin glycoproteins, bovine mucin glycoproteins, or human mucin glycoproteins.

In various embodiments, the multi-arm thiol functional cross-linker comprises a 4-arm polyethylene glycol thiol of the structure

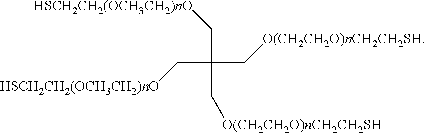

The mucin glycoproteins cross-linked in the synthetic hydrogel matrix by such multi-arm thiol functional cross-linker may be of any suitable type, and may for example be porcine mucin glycoproteins or bovine mucin glycoproteins.

The synthetic hydrogel as variously described herein may have properties that are similar to natural mucus. For example, the synthetic hydrogel may have an elastic modulus G′ of from 100 to 400 Pa in an angular frequency range of 0.1 to 200 rad/sec, and a viscous modulus G″ of from 3 to 90 Pa in an angular frequency range of 0.1 to 200 rad/sec, when measured at pH 7.4 and 37° C.

Thus, in various embodiments of the present disclosure, the synthetic hydrogel may be characterized by the following characteristics:
(i) the multi-arm thiol functional cross-linker comprises a 4-arm polyethylene glycol thiol of the structure

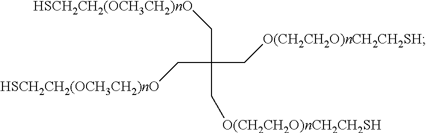

(ii) the mucin glycoproteins are porcine mucin glycoproteins or bovine mucin glycoproteins; and
(iii) the synthetic hydrogel has an elastic modulus G′ of from 100 to 400 Pa in an angular frequency range of 0.1 to 200 rad/sec, and a viscous modulus G″ of from 3 to 90 Pa in an angular frequency range of 0.1 to 200 rad/sec, when measured at pH 7.4 and 37° C.

The present disclosure in another aspect relates to a method of making a synthetic hydrogel, comprising:
combining mucin in aqueous medium with a multi-arm thiol functional cross-linker; and
cross-linking the mucin with the multi-arm thiol functional cross-linker to form the synthetic hydrogel.

In such method, the aqueous medium may be of any suitable type, and may for example comprise a buffered aqueous medium. The mucin employed in such method may likewise be of any suitable character, e.g., porcine mucin or bovine mucin, in dry powder form.

Consistent with the foregoing discussion, the method may be carried out to form a synthetic hydrogel of appropriate character, e.g., having an elastic modulus G′ of from 100 to 400 Pa in an angular frequency range of 0.1 to 200 rad/sec, and a viscous modulus G″ of from 3 to 90 Pa in an angular frequency range of 0.1 to 200 rad/sec, when measured at pH 7.4 and 37° C.

In various embodiments, the method is carried out to form the synthetic hydrogel, wherein the multi-arm thiol functional cross-linker comprises a 4-arm polyethylene glycol thiol of the structure

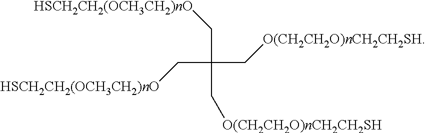

The present disclosure reflects the discovery that disulfide cross-links mediate gel formation and that chemical treatments that block or reduce cysteines result in inhibition or disruption, respectively, in mucin hydrogel network formation. Utilizing particle tracking microrheology to investigate the kinetics and evolution of microstructure and viscoelasticity within hydrogel during its formation, it has been found that gel formation rate can be tuned by varying mucin to cross-linker ratio, to achieve network pore sizes in the range previously measured for human mucus, thereby enabling the manufacture of mucin hydrogels with physiologically relevant properties using readily available reagents in a simple and cost-effective manner.

The present disclosure provides a simple innovative approach to generate hydrogels using crude, commercially available mucins and thiol-based cross-linkers that re-establish the native rheological properties of such mucins, by assembling such mucins into mucus gels.

In the development of this approach, particle tracking microrheology (PTM) of PEG-coated nanoparticles was employed to determine if and when mucins began to form into a gel after addition of a cross-linking reagent, and thiol-functionalized, polymer-based crosslinkers with varying chemistry and geometry were tested to identify cross-linking reagents that would initiate mucin hydrogel network formation.

Using an optimized cross-linking strategy, bulk rheological assessment of mucin-based hydrogels was performed to determine the ability to produce gels with physiological viscoelastic properties, and PTM was utilized to investigate the kinetics of gel formation as a function of mucin and cross-linker concentration. As a result of this effort, the present inventors have demonstrated that crude, commercially available mucins, which as supplied are incapable of forming viscoelastic gels, may be restored to behave like natural mucus.

Accordingly, the present disclosure contemplates a synthetic mucin hydrogel that is formed from mucin that is combined in aqueous medium with a thiol-functional cross-linker, e.g., a multi-arm thiol functional cross-linker, or other cross-linker with thiol functionality, to form the mucin-based hydrogel material. The mucin raw material for synthesis of the hydrogel may be of any suitable type and may for example comprise porcine mucin, bovine mucin, human mucin, or other animal-derived mucin, or may comprise synthetic mucin material, or may comprise a combination of natural and synthetic mucins. The thiol functional cross-linker likewise may be of any suitable type that is effective in interaction with the mucin raw material in aqueous medium to form the synthetic mucin hydrogel of desired character.

As discussed hereinabove, the thiol functional cross-linker may comprise a multi-arm thiol functional cross-linker. As previously mentioned, the multi-arm thiol functional cross-linker may be a multi-arm cross-linker with thiol functionality at the termini of each of its arms. The multi- arm thiol functional cross-linker may have 3 or more arms, e.g., from 3 up to 10 or more arms, with arms radiating from a central hydrocarbyl moiety or other organic core structure of appropriate character, or with arms constituted in a dendrimeric, oligomeric, or other multimeric structure, wherein multiple arms are terminated at outer extremities thereof with thiol functionality. The arms may comprise or be primarily constituted by hydrophilic chains, e.g., polyalkyloxy linear segments with terminal thiol (—SH) functionality. In particular embodiments, the arms may comprise chains of the formula —O(CH₂CH₂O)ₙCH₂CH₂SH wherein n is at least 1 and may be up to 10, 20, 50, 100, 500, 1000, 10,000 or more, as appropriate to the specific mucin hydrogel synthesis that is involved. In particular embodiments, the respective arms in the multi-arm cross-linker structure may be linked with one another by a central alkyl, alkoxy, quaternary ammonium, or other organo-core structure, e.g., tert-butyl, tert-pentyl, tetrabutylammonium, tris(tert-pentoxy), etc.

In an illustrative implementation, a gastric mucin such as porcine gastric mucin (PGM) may be processed with a 4-arm polyethylene glycol thiol of the formula

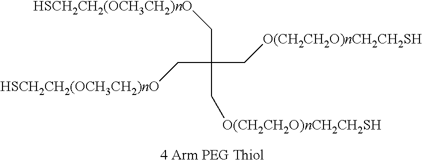

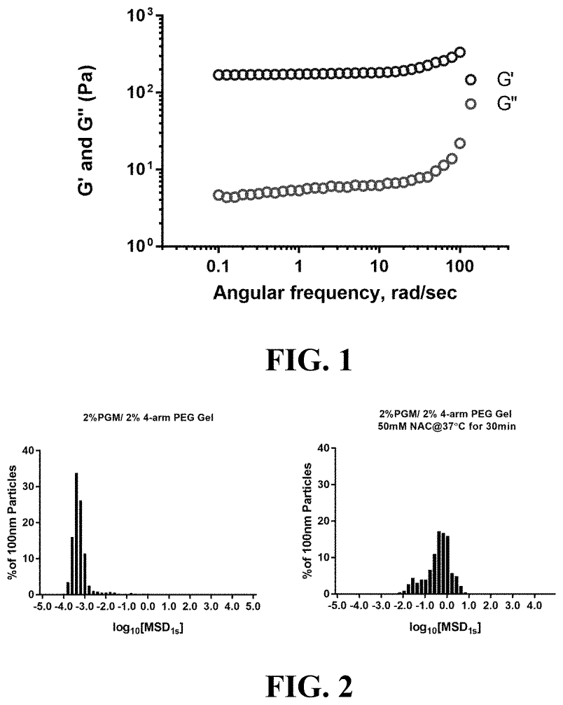

to form a mucin-based hydrogel material, e.g., a 2% PGM 2% 4-arm-PEG-SH hydrogel whose G′ and G″ characteristics are shown in FIG. 1, which is a graph of G′ and G″ as a function of angular frequency, rad/sec, for a frequency sweep of the 2% PGM 2% 4-arm-PEG-SH after 12 hours at pH 7.4. At an angular frequency of 1 rad/sec, G′≈175 Pa and G″≈5.3 Pa.

The microrheology of such illustrative 2% PGM 2% 4-arm-PEG-SH hydrogel is shown in FIG. 2, wherein the percentage of 100 nm PEG-coated polystyrene particles used for such determination is shown as a function of log₁₀[MSD₁ₛ] mean squared displacement of such particles after 12 hours in the hydrogel (graph a), and in which the mean squared displacement of the particles in the hydrogel is shown after 30 minutes treatment of the hydrogel with mucolytic N-acetylcysteine (NAC).

Such illustrative example demonstrates the ability to form mucin-based hydrogels based on disulfide linkages. It will be appreciated that the properties of such mucin-based hydrogels may be selectively varied by use of appropriate 4-arm-PEG-thiol cross-linker concentrations, or other thiol-functional cross-linkers, to provide mucin-based hydrogels with desired properties for particular end-use applications.

Accordingly, the present disclosure contemplates the design of mucin-based hydrogels using cross-linkers capable of forming disulfide linkages between mucins, to produce mucin-based hydrogels possessing viscoelastic properties of desired character, e.g., mucin-based hydrogels with viscoelastic properties similar to natural mucus as measured by bulk rheology. Particle tracking microrheology may be employed to investigate the kinetics and evolution of microstructure and viscoelasticity within the gel during its formation, as well as in the final product hydrogel, and gel formation can be tuned by appropriate selection of the mucin to cross-linker ratio.

The present disclosure in a further aspect relates generally to a system and method for microrheology, which enables the measurement of the rheological properties of complex fluids using a microplate reader, in a simple, direct, and cost-effective manner.

In such aspect, the disclosure provides a method of microrheologically characterizing mucus, comprising:

dispersing in the mucus muco-inert particles (MIP);

irradiating the mucus containing MIP with polarized light; and

measuring fluorescence polarization (FP) resulting from rotational diffusion of the MIP in the mucus in response to such irradiating, as a microrheological characteristic of the mucus.

Such method may be carried out, wherein the mucus containing MIP is formed in or introduced to a well of a plate, and the FP is measured using a plate reader equipped with a spectrofluorometer and polarized filter set, to which the plate having the mucus containing MIP in the well thereof is introduced for the measuring.

Accordingly, the disclosure contemplates a system for microrheologically characterizing mucus, comprising muco-inert particles (MIP) of appropriate character for the microrheological characterization, one or more single well or multiwell plates, and a plate reader equipped with a spectrofluorometer and polarized filter set, to which such single well or multiwell plates containing mucus and MIP therein can be introduced for irradiating the MIP-containing mucus and measuring fluorescence polarization (FP) resulting from rotational diffusion of the MIP in the mucus in response to such irradiating.

In the above-described method conducted in such system, the method may further comprise determining microviscosity of the mucus based on the measured FP, as hereinafter more fully described.

The MIP used in the foregoing system and method may be of any suitable type appropriate for the FP determination, and may for example comprise MIP have a size in a range of from 50 nm to 1000 nm. In various embodiments, the MIP used in such system and method may comprise polymeric nanoparticles, of polystyrene or other suitable material, that are coated with a mucus adhesion-resistant coating.

The above-described system and method may be utilized in various implementations for microrheological characterization of mucus, to identify presence, absence, progression, or prognosis of obstructive lung disease.

In mucus microrheology applications, the system and method of the present disclosure enable highly sensitive, functional assessment of mucus properties to be performed in clinical settings using equipment that is readily available in such facilities. By avoiding the need for complex, application-specific equipment to perform microrheological measurements, the system and method of the present disclosure achieve a major advance in the art, and broaden the availability and utility of microrheology as a fundamental tool for materials characterization and biomedical applications.

The microrheology system and method of the present disclosure reflects the discovery that the rotational diffusion of muco-inert nanoparticles (MIP), as measured by fluorescence polarization (FP) using existing equipment and technology commonly found in clinical laboratories, correlates with MIP translational diffusion and is sensitive to mucus microrheology. Based on this discovery, a method has been developed to estimate microviscosity from FP-based rotational diffusion measurements. This microviscosity quantitation method is more fully described in the ensuing disclosure, using illustrative glycerol:water mixtures of known viscosity.

While specifically disclosed in application to characterization of mucus in the ensuing disclosure, it will be recognized that the microrheology system and method of the present disclosure can be applied to a vast spectrum of biological as well as non-biological materials. Those of ordinary skill in the art can readily apply the techniques illustratively described herein, based on the present disclosure, to characterize such materials with respect to their microrheological properties in particular applications relevant to such materials.

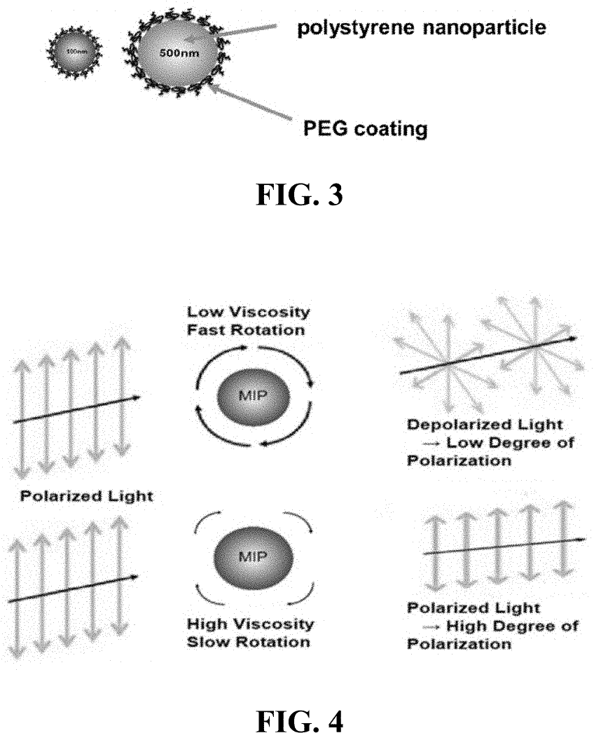

By way of illustration, the characterization of mucus may be carried out using muco-inert particles (MIP) produced by coating a dense layer of polyethylene glycol (PEG) or other suitable coating material to the surface of nanoparticles, e.g., polystyrene or other polymeric nanoparticles of appropriate diameter, such as 100 nm and 500 nm polystyrene nanoparticles shown in FIG. 3. Using such MIP, the rotational diffusion of the MIP can be measured using fluorescence polarization (FP). FP is commonly used in clinical laboratories to perform immunoassays and blood, urine, and other clinical sample materials.

As shown in FIG. 4, the polarization value of the MIP will be dependent on the size of the MIP and the viscosity of the sample. When the MIP is in a low viscosity fast rotation regime, the interaction of the MIP with polarized light will produce depolarized light with a low degree of polarization, and when the MIP is in a high viscosity slow rotation regime, the interaction of the MIP with polarized light will produce polarized light with a high degree of polarization, and the rotational diffusion of the MIP can be correspondingly measured by fluorescence polarization techniques.

Although prior work of the present inventors and others have demonstrated how altered mucus properties significantly contribute to disease pathology, there are currently no clinically employed techniques to examine the biophysical features of mucus produced by patients. In this context, the present nanoparticle-based method achieves a substantial advance in the art, enabling assessment of the biophysical properties of mucus to provide clinically relevant information on patient disease status.

The mucus microrheology assessment methods and systems of the present disclosure facilitate the use of MIP-based mucus biomarkers in hospitals and other clinical settings, for nanobiotechnological performance of highly sensitive, functional assessments of mucus properties, utilizing equipment that is readily available in such facilities, without the necessity of specialized equipment that has heretofore been used in mucus analysis in research studies. By using the mucus microrheology assessment methods and systems of the present disclosure, clinicians can quantitatively monitor the properties of airway mucus and examine how it relates to disease progression and/or the efficacy of therapeutics aimed at restoring mucus function.

The references of Fahy, J. V. & Dickey, B. F. Airway mucus function and dysfunction. The New England journal of medicine 363, 2233-2247, doi:10.1056/NEJMra0910061 (2010), Duncan, G. A. et al. Microstructural alterations of sputum in cystic fibrosis lung disease. JCI Insight 1, doi:10.1172/jci.insight.88198 (2016), and Shinitzky, M. & Barenholz, Y. Fluidity parameters of lipid regions determined by fluorescence polarization. Biochimica et Biophysica Acta (BBA)—Reviews on Biomembranes 515, 367-394, doi:https://doi.org/10.1016/0304-4157(78)90010-2 (1978) are incorporated herein by reference in their entireties, except for any statements contradictory to the express disclosure herein, subject matter disclaimers or disavowals, and except to the extent that the incorporated material is inconsistent with the express disclosure herein, in which case the language in this disclosure controls. Incorporation by reference of such materials shall not be considered an admission by the applicant that the incorporated materials are prior art to the present disclosure, nor shall any of such referenced documents by reason of such incorporation be considered material to patentability of the present disclosure.

The features, advantages, and characteristics of the synthetic hydrogels and preparative techniques of the present disclosure will be more fully appreciated in the context of the following, non-limiting examples.

### EXAMPLE 1

Nanoparticle Preparation

Carboxylate modified fluorescent PS spheres (PS-COOH; Life Technologies) with a diameter of 100 nm or 500 nm were coated with a high surface density of polyethylene glycol (PEG) via a carboxyl-amine linkage using 5-kDa methoxy PEG-amine (Creative PEGWorks). PEG-amine was added to a diluted suspension of PS-COOH in ultrapure water N-hydroxysulfosucciniminde sodium salt (10 mM; Alfa Aesar), borate buffer (pH 8.3) and 1-ethyl-3-(3-dimethylaminopropyl) carbodiimide hydrochloride (2 mM; Thermo Fisher) were then added to activate and link PEG-amine to PS-COOH nanoparticles. The reaction was mixed for at least 4 hours at room temperature and covered to minimize exposure to light. After mixing, excess reagents were removed by washing and centrifuging three times with ultrapure water, then re-suspended to a final volume two times the original. After washing, particle size and zeta potential was measured in 10 mM NaCl at pH 7 using a Malvern Zetasizer Nano Z590. Measured diameters of 122 nm and 428 nm and zeta potentials of −4.22 and −3.4 mV were recorded for 100 nm and 500 nm PEG-coated PS nanoparticles, respectively.

Mucin-Based Hydrogel Preparation

Solutions of porcine gastric mucin (PGM; Sigma-Aldrich) or mucin from bovine submaxillary glands (BSM; Sigma-Aldrich) were prepared in a physiological buffer (154 mM NaCl, 3 mM CaCl₂, and 15 mM NaH₂PO₄ at pH 7.4) and stirred at room temperature for 2 hours. Crosslinking reagents tested in this work included monofunctional PEG-thiol (PEG-1SH; 10 kDa), bifunctional PEG-thiol (PEG-2SH; 10 kDa), 4-arm PEG-thiol (PEG-4SH; 10 kDa), each purchased from Laysan Bio Inc. and a thiol-functionalized dextran (dextran-SH; 10 kDa; ˜60-120 thiol groups prepolymer) purchased from Nanocs. Cross-linking solutions were prepared separately in buffer directly before mixing with mucin solutions. To initiate gelation, equal volume aliquots of each solution were mixed and either equilibrated for 30 minutes before particle tracking or left for 24 hours before bulk rheological characterization at room temperature. To confirm cross-linker interaction with cysteine rich regions of PGM, thiol-blocking and disulfide cleavage experiments were performed to inhibit or disrupt gel assembly. Iodoacetamide at 50 mM (VWR) was added to the PGM solutions and mixed for 1 hour to alkylate PGM cysteine residues before mixing with the crosslinking polymer. Disulfide bonds within the gel were disrupted using the reducing agent, N-acetyl cysteine (NAC). NAC was added after gelation (>9 hours) at a final concentration of 50 mM (NAC) and incubated at 37° C. for 30 minutes.

Cysteine content in PGM and BSM was measured using a previously described fluorometric assay (Yuan, S.; Hollinger, M.; Lachowicz-Scroggins, M. E.; Kerr, S. C.; Dunican, E. M.; Daniel, B. M.; Ghosh, S.; Erzurum, S. C.; Willard, B.; Hazen, S. L.; Huang, X.; Carrington, S. D.; Oscarson, S.; Fahy, J. V., Oxidation increases mucin polymer cross-links to stiffen airway mucus gels. Science translational medicine 2015, 7, 276ra27). Briefly, 50% w/v solutions of PGM and BSM were prepared. The solutions were then diluted 10-fold from the original volume in 8 M guanidine-HCl (Sigma Aldrich). Serial dilutions of 5 mM L-cysteine (Sigma-Aldrich) in 50 mM tris-HCl were used as a standard. Samples and standards were added to a black Maxisorp plate with an equal volume of 2 mM monobromobimane (mBBr, Sigma Aldrich) in 50 mM tris-HCl. The fluorescence was measured at excitation and emission wavelengths of 395 and 490 nm, respectively. Based on this analysis, 0.18 μM and 0.16 μM cysteine content per mg mucin was measured for PGM and BSM, respectively.

Bulk Rheological Measurements

Dynamic rheological measurements of the mucin/PEG-4SH hydrogels were performed using an AR2000 rheometer (TA Instruments) with a 20-mm diameter parallel plate geometry at 25° C. To determine the linear viscoelastic region of the fully formed gel, a strain sweep measurement was collected from 0.1-10% strain at a frequency of 1 rad/s. To determine the elastic modulus, G′(ω), and viscous modulus, G″(ω), a frequency sweep measurement was conducted within the linear viscoelastic region of the gel, at 1% strain amplitude and angular frequencies from 0.1 to 100 rad/s.

Sample Preparation for Fluorescent Video Microscopy

The diffusion of PEG-coated nanoparticles (PEG-NP) in mucin-based hydrogels were measured using fluorescent video microscopy. Samples were prepared with 0.5 μL of 0.002% w/v suspension of PEG-NP, added into a 25 μL solution of PGM or BSM and cross-linker prior to gel formation. Next, ˜25 μL of mucin hydrogel precursor solution containing PEG-NP was added into a custom microscopy chamber, sealed with a cover slip, and equilibrated for 30 minutes at room temperature before imaging. Fluorescent video microscopy experiments were collected using a Zeiss 800 LSM microscope equipped with a x63/1.20 W Korr UV VIS IR water-immersion objective and an Axiocam 702 camera with pixel resolution of 0.093 μm/pixel. Images were collected at a frame rate of 33.33 Hz for 10 seconds at room temperature. For studies of gel kinetics, videos were taken every hour in order to measure PEG-NP diffusion during hydrogel assembly.

Particle Tracking Microrheology (PTM)

The particle tracking data analysis used in this work was based on a previously developed image processing algorithm. The image intensity threshold was set to differentiate nanoparticle signal from background. Nanoparticles above the intensity threshold were then assigned centers based on x and y image location. Particles were tracked for at least 300 sequential frames, allowing trajectories to be determined. From these trajectories, time-averaged mean squared displacement (MSD; <Δr²(r)> as a function of lag time, τ, was calculated as <Δr²(τ)>=<[x(t+τ)−x(t)]²+[y(t+τ)−y(t)]²>.

Using the generalized Stokes-Einstein relation, measured MSD values were used to compute viscoelastic properties of the hydrogels. The Laplace transform of <Δr²(r)>, <Δr²(s)>, is related to viscoelastic spectrum \tilde{G}(s) using the equation \tilde{G}(s)=2k_{B}T/[πas<Δr²(s)>], where k_{B}T is the thermal energy, a is the particle radius, and s is the complex Laplace frequency. The complex modulus can be calculated as G*(ω)=G′(ω)+G″(iω), with iω being substituted for s, where i is a complex number and ω is frequency. Hydrogel network pore size, is estimated based on MSD using the equation, ξ≈\sqrt{<Δr²(τ)>}+a, and also based on G′ using the equation, ξ≈(k_{B}T/G′)^{1/3}. The sol-gel transition (gel point) was determined by calculating the logarithmic slope of the mean-squared displacement, α=log₁₀(<Δr²(τ)>)/log₁₀(τ). A critical value of n=0.5 was used, where the gel point is defined as the time when measured α<n.

Design and Characterization of a Mucin-Based Hydrogel Network Assembly

A viscoelastic gel composed of porcine gastric mucin (PGM) via disulfide-bond mediated crosslinking between the cysteine domains of PGM biopolymers was sought to be created. PTM was employed to evaluate different strategies to make mucin-based gels. PTM was selected since it provides a biophysical method to assess mechanical properties of soft biological gels, and has been used extensively in previous work to evaluate microrheology and structure of mucus. In comparison to bulk rheological methods, PTM can provide additional information on material properties on the micro- to nanoscale, such as network pore size and local heterogeneity.

The diffusion of fluorescently labeled, PEG-coated particles (PEG-NP) with diameter of 100 nm was measured in mixtures of PGM and various thiol-containing polymers to determine which may be a suitable cross-linking reagent. A low concentration (˜4×10⁻⁵% w/v) of nanoparticles coated with a dense layer of low molecular weight PEG was used in order to ensure that nanoparticle probes were minimally adhesive to mucins, and would be unlikely to interfere with or influence hydrogel formation.

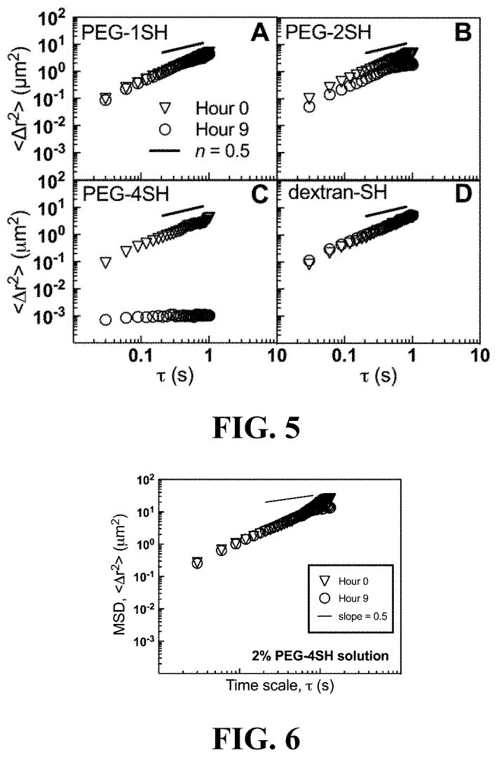

FIG. 5 shows PEG-NP diffusion as measured by the mean squared displacement (MSD; (<Δr²>) of the particles determined immediately after mixing (hour 0) and 9 hours after mixing. Once the gel is formed, the MSD of PEG-NP is reduced due to steric interactions with the mesh-like mucin polymer network. In addition, the logarithmic slope of MSD is reduced from ˜1 for a viscous liquid to ≤0.5 for a viscoelastic material.

Four potential thiol-based crosslinking reagents were tested with a low molecular weight of 10 kDa and a PEG or dextran backbone. Two percent w/v of mucin and crosslinker was used in each case. At these concentrations, the number of thiols in the crosslinkers (1-10 mM for PEG; 120-140 mM for dextran) is far in excess of that of the mucin biopolymers (2-10 μM range) in all cases. As shown in FIG. 5, in panel A, no significant changes in MSD of PEG-NP were observed after mixing PGM with monofunctional PEG-1SH. Since it is monofunctional, it is not able to initiate gel formation. It was hypothesized that bifunctional PEG-2SH would be able to create disulfide bridges between mucin polymers to facilitate gel formation. However, it was found to be unable to form a gel over a 9-hr period (FIG. 5, panel B) Using a multi-arm PEG-4SH, a substantial decrease in the magnitude and slope of MSD was observed after 9 hours of incubation with PGM, indicative of network formation (FIG. 5, in panel C). Concerning the specific action of the PEG linker geometry on mucin hydrogel assembly, it can be hypothesized that the branched architecture of PEG-4SH enabled association between multiple mucin chains, whereas PEG-2SH mediated bonds between 2 neighboring chains may only effectively increase polymer weight in semidilute solution of mucins.

Another complexity in this biohybrid system is discerning the relative contributions of PEG-PEG, mucin-mucin, and PEG-mucin associations to the sol-gel transition. To address this, the potential self-assembly of PEG-4SH was considered, yielding a hydrogel that does not incorporate mucin biopolymers. The microrheology of a 2% PEG-4SH solution (in the absence of PGM) was first measured, and no gel formation was observed in a 9-hour period (FIG. 6).

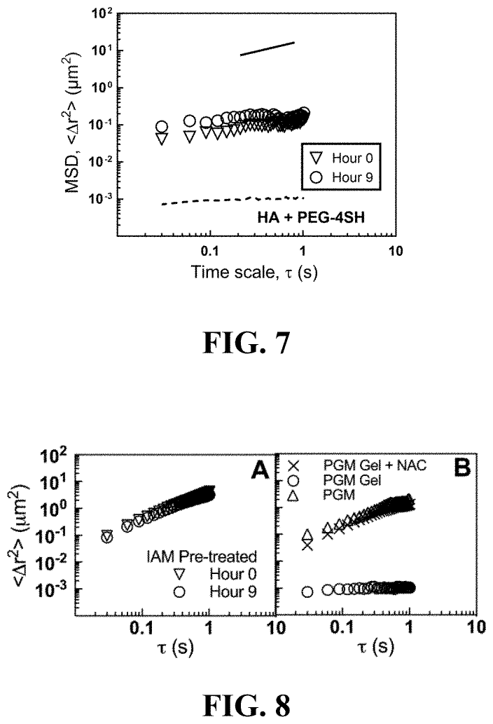

The issue of whether PEG-4SH self-assembly could be the result of an increased local concentration due to crowding by mucin polymers was also considered. To evaluate this, 500 kDa hyaluronic acid (HA) was used to act as a non-thiol containing, high molecular weight crowding agent. Hydrogel network formation was not observed after a 9-hour period in mixtures of 2% HA and 2% PEG-4SH (FIG. 7), suggesting that crowding did not induce PEG-4SH self-assembly. The cross-linking polymer backbone then was changed from PEG to dextran, but unlike PEG-4SH, the multi-functional dextran-SH did not lead to assembly of mucins into a mesh network (FIG. 5D). These results suggested that the more flexible PEG backbone may also be important in the ability of PEG-4SH to facilitate hydrogel formation. Based on these results, PEG-4SH was selected as the optimal cross-linking polymer to produce mucin-based hydrogels.

In order to examine the mechanism of gel formation, the impact of alkylation or reduction of cysteines on the formation of mucin-based hydrogels was evaluated. PTM was again employed to monitor changes in the mucin hydrogel network upon treatment with cysteine-blocking or disulfide bond disrupting agents. In order to block available cysteines, a 2% PGM solution was alkylated with iodoacetamide (IAM). While only the PGM solution was treated with IAM, it was noted that excess IAM would most likely block cysteines present on PEG-4SH once the solutions were mixed. As shown in FIG. 8, no significant changes in 100 nm PEG-NP diffusion were observed after 9 hours, implying that network assembly was inhibited by IAM treatment. To determine if the hydrogel network would be disrupted by cleaving disulfide bonds, the reducing reagent, N-acetyl cysteine (NAC), was applied to a fully formed PGM/PEG-4SH hydrogel. After 30-minute treatment with NAC, a marked increase in 100 nm PEG-NP diffusion approaching that of the PG-M solution alone was observed (FIG. 8 in panel B), indicative of a significant breakdown in the mesh network. Together, these data further support that disulfide bonds between PGM and PEG-4SH drive network assembly.

Macro- and Microrheology of Mucin-Based Hydrogels

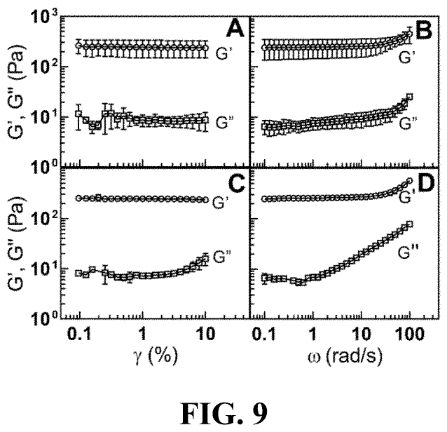

To determine if a viscoelastic gel similar to native mucus was formed, the bulk elastic (G′) and viscous (G″) moduli of 2% PGM after mixing and incubation for 24 hours with 2% PEG-4SH were measured. Strain sweep experiments were performed at frequency of ω=1 rad/s from 0.1 to 10% strain as shown in FIG. 9 in panel A. Elastic solid-like behavior of the material was observed with G′>G″ and loss tangent (tan δ) of 8.1, thus confirming the viscoelastic nature of the hydrogel. No evidence of yielding was found and thus, results were within the linear viscoelastic region of the gel. To determine if the behavior of the hydrogel was frequency dependent, a frequency sweep was performed at 1% strain over a physiologically relevant frequency range of 0.1-100 rad/s. The hydrogel was confirmed to be stable over the full range of frequencies tested (FIG. 9 in panel B). Importantly, it was found that the elastic modulus of ˜244 Pa at ω=1 rad/s is within the physiological range for mucus. These results suggest that PEG-4SH is able to support gelation of mucins into a viscoelastic network. To determine if this approach was generalizable to other mucin types, a hydrogel was prepared using another commercially available mucin, bovine submaxillary mucin (BSM). The bulk elastic (G′) and viscous (G″) moduli of 2% BSM after mixing and incubation for 24 hours with 2% PEG-4SH were measured. Formation of a stable viscoelastic hydrogel was observed, with G′ >G″ and a loss tangent (tan 6) of 1.4 (FIG. 9, in panels C, D).

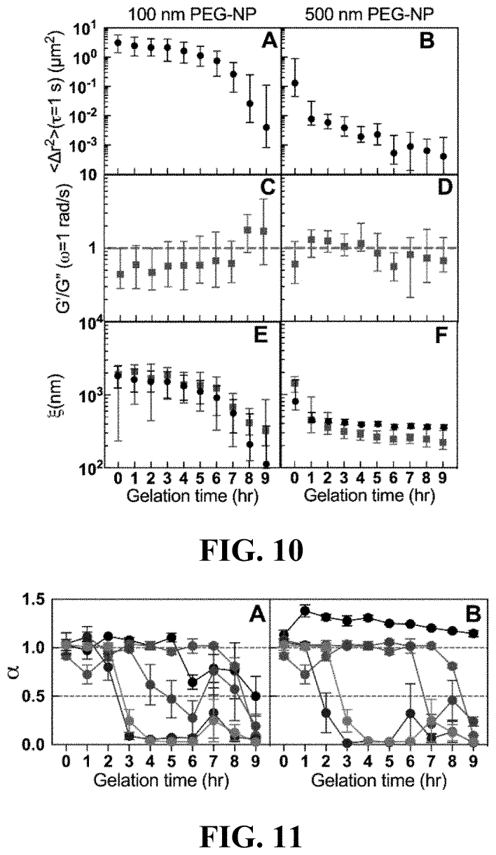

Next, PTM was used to gain additional insights into the biophysical properties of the material. Given previous reports showing native mucus having network pore sizes ranging from 100-500 nm, microrheology of a 2% PGM and 2% PEG-4SH hydrogel was measured using PEG-NP probes with 100 and 500 nm diameter. PTM measurements were performed each hour for a total of 9 hours to capture local changes in viscosity caused by the organization of mucin fibers into a hydrogel network over time. FIG. 10 in panels A and B shows MSD at τ=1 s for 100 nm and 500 nm PEG-NP, respectively. A decrease in MSD as a function of gelation time was observed for both the 100 and 500 nm PEG-NP, indicating a decrease in diffusion rate as a result of gel network formation. Next, the ratio of elastic and viscous moduli (G′/G″) was calculated based on 100 nm (FIG. 10, in panel C) and 500 nm (FIG. 10, in panel D) PEG-NP probes. It should be noted these local measurements of G′ and G″ will strongly depend on probe size as the gel network evolves over time. Based on measured MSD and G′, approximate hydrogel network mesh pore sizes were found to be on the order of microns at hour 0 and steadily decreasing with median pore sizes on the order of 200-300 nm, consistent with findings in mucus collected from humans.

Using nanoparticle probes of different size enabled resolution of the early and late stages of mucin hydrogel assembly. Based on this analysis, evidence of distinct phases in mucin organization into a hydrogel network (i.e., transitions in G′/G″ to values >1) was found. After ˜1 hour, 500 nm probes detected an initial rise in elasticity and reduction of network pore sizes (FIG. 10, in panels D and F). Starting at approximately hour 7, further reductions in network pore size are evident based on the relative increase in G′/G″ measured by 100 nm probes (FIG. 10, in panels C and E). These data may indicate that the mucin hydrogel forms with a rate of gelation that slows as the network pore dimensions decrease.

Concentration Dependence of the Sol-Gel Transition

Gelation kinetics were considered in connection with manipulation of the sol-gel transition of the hydrogels.

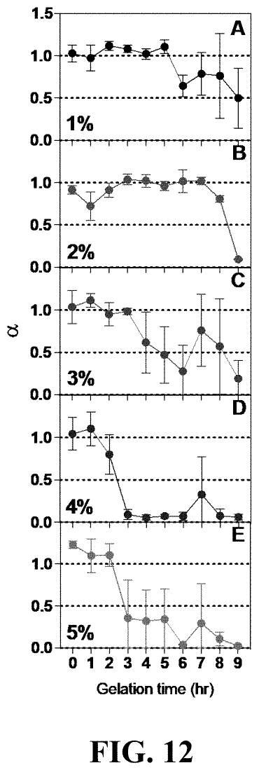

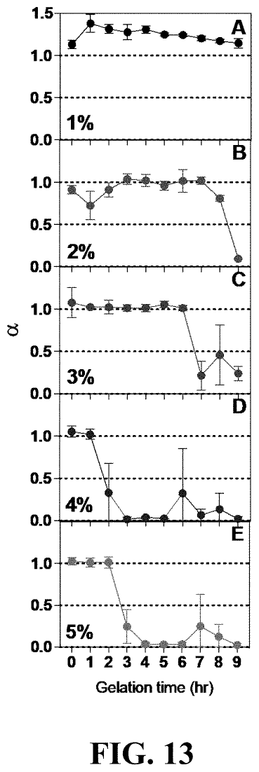

To understand the influence of mucin to crosslinker ratio on gel assembly, PGM and PEG-4SH concentration were systematically varied to assess their impact on gelation kinetics. The time of gelation was determined based on measured MSD of 100 nm PEG-NP defined as α=log₁₀[MSD]/log₁₀[τ]=0.5. Given that 100 nm PEG-NP captured the latter stages of gel assembly (FIG. 10), probes of this size were used to monitor changes in a every hour for 9 hours as the network assembled. PGM concentration was varied 1-5% w/v while keeping the PEG-4SH concentration constant at 2% w/v. The data in FIG. 11 in panel A show that increasing PGM concentration leads to increases in the rate of gelation evident by measured a values. Next, PEG-4SH concentration was varied from 1-5% w/v, while keeping PGM concentration constant at 2% w/v. A concentration dependent change in gelation kinetics was observed, with a reduction in the time needed to form a gel as PEG-4SH concentration is increased (FIG. 11 in panel B). Each series of results with data from individual experiments are provided in FIGS. 12 and 13.

These results are consistent with our earlier findings showing that the gelation process is dependent on both PGM and PEG-4SH to create the viscoelastic gel. From control experiments, no evidence of gel formation was found in solutions of 1-5% PGM (in the absence of PEG-4SH) and 1-5% PEG-4SH (in the absence of PGM) after a 9-hr period (FIGS. 12 and 13). These data suggest variation in gelation kinetics is likely dependent on bond formation between mucins and PEG-4SH. As one would correspondingly intuitively expect, increasing the concentration of either mucin or crosslinking polymers leads to a decrease in the time required to form the gel. Starting from either 1% PGM or 1% PEG-4SH appears to be below the threshold concentration required to form a gel within a 9-hour period, although 1% PGM does approach the gel point near hour 9. As PGM concentration increases, a steady decrease in gelation time is observed from an initial gel point of ˜9 hours for 2% w/v PGM, to ˜5 hours for 3% w/v PGM and to ˜3 hours for 5% w/v PGM (FIG. 11, in panel A) This can be simply explained by the observation that as PGM concentration increases, the closer proximity between fibers makes PEG-4SH-mediated bridging more probable and as a result, gel assembly is more rapid. Interestingly, a more step-wise response in gelation time is observed as a function of PEG-4SH concentration with ˜8-9 hour for the lower 2% and 3% PEG-4SH concentrations and much faster ˜2-3 hr gelation times for the higher 4% and 5% PEG-4SH concentrations (FIG. 11, in panel B). This might be explained by PEG-4SH attaching to the mucin fibers at higher density, with the rate of assembly being enhanced due to an increase in their multi-valent bridging capacity. The observed increase in gelation time at 4% compared to 5% was attributed to slight variation and start time between experiments and the relatively low resolution in gelation time.

The foregoing results demonstrate how the mucin-based hydrogel of the present disclosure may be tuned to assemble in a user-defined timeframe. This presents an opportunity for the hydrogel system to be designed for various biomedical applications where slower kinetics (e.g., cell encapsulation) or more rapid kinetics (e.g., injectable gels) may be desirable. The hydrogel system of the present disclosure may also be used for fundamental studies on the role of kinetics in mucus gel formation and its biological function, which due to the lack of available models has not been previously investigated.

The foregoing empirical results show that a mucin-based synthetic hydrogel of highly useful character has been produced and may be quantitatively characterized using particle tracking microrheology. It has been shown that hydrogel assembly is mediated by disulfide linkages, in the illustratively disclosed synthetic hydrogels formed by porcine gastric mucin and 4-arm PEG-thiol polymers. Bulk rheology confirms the formation of the mucin-based synthetic hydrogel as a solid-like viscoelastic material with an elastic modulus resembling that of naturally produced mucus. During the assembly process, the evolution of microscale structure in the mucin gel network has been shown by microrheological measurements using PEG-coated nanoparticle probes of different sizes. It has been shown that gelation kinetics can be tailored by changing mucin or cross-linker concentrations.

Based on the above-described empirical results and preceding disclosure, it will be apparent that those of ordinary skill in the art may readily optimize the mucin-based hydrogel for specific end-use applications, and determine the appropriate processing conditions and hydrogel characteristics appropriate to such applications, e.g., by varying of buffer conditions such as ionic strength and pH, and selecting other appropriate processing conditions, to provide mucin-based hydrogels of corresponding desired properties of tissue adhesion, pathogen capture, and transportability of the gel in response to shear, which are useful for a wide range of biological and medical applications.

### EXAMPLE 2

Preparation of MIP

Muco-inert nanoparticles (MIP) were formulated using commercially available fluorescent polystyrene (PS) nanoparticles with diameters ranging from 50-1000 nm, following the procedure described in Duncan, G. A. et al. Microstructural alterations of sputum in cystic fibrosis lung disease. JCI Insight 1, doi:10.1172/jci.insight.88198 (2016). Specifically, PS particles were covalently coated using NHS ester chemistry with 5 kDa PEG at high densities required to make particle surfaces resistant to adhesion to the mucus gel network. A mucus hydrogel model was constructed by mixing varying w/v concentrations of porcine gastric mucin (PGM) with 2% w/v 4-arm PEG-thiol (PEG-45H; 10 kDa) to enable cross-linking into a gel with physiological viscoelastic properties.

Determination of Diffusional and Polarization Characteristics of MIP

Translational and rotational diffusion of MIP were evaluated using PTM and FP, respectively. For PTM, 25 μL aliquot of the mucus hydrogel were mixed with 0.5 μL solution containing MIP and transferred to custom-made chambers for imaging. Twenty-second movies at 30 ms temporal resolution were acquired with a high-speed CMOS camera equipped on an inverted confocal microscope with a 63×/1.4 NA oil objective. Movies were analyzed using tracking software in MATLAB to extract 2D x, y-coordinates of MIP centroids over time. From these trajectories, time averaged mean squared displacement (MSD), an indicator of translation diffusion, for individual particles were determined.

For FP, 50 μL aliquot of the mucus model were mixed with 1 μL solution containing MIP and transferred to a multi-well maxi-sorb plate. FP was measured using a plate reader equipped with a spectrofluorometer and polarized filter set. Polarization (P) of the fluorescence signal was calculated as, P=[F_{∥}−F_{⊥}]/[F_{∥}+F_{⊥}], where F_{∥} and F_{⊥} are the fluorescence intensity parallel and perpendicular to the excitation plane, respectively, and are reported in units of mP where 1000 mP=1 P.

Determination of Microviscosity from FP

The method to calculate microviscosity from FP-based MIP rotational diffusion measurements has been adapted from an approach described in Shinitzky, M. and Barenholz, Y., Fluidity parameters of lipid regions determined by fluorescence polarization, Biochimica et Biophysica Acta (BBA)—Reviews on Biomembranes 515, 367-394, doi:https://doi.org/10.1016/0304-4157(78)90010-2 (1978), in which FP was used to measure the fluidity of lipid membranes using the equation

$$η = \frac{{C}_{r} T τ r}{{r}_{0} - r} \qquad ( 1 )$$

where η is the calculated microviscosity in units of poise, Cᵣ is a parameter that is dependent on the shape of the fluorescently labeled probe, T is the absolute temperature, τ is the lifetime of the fluorophore, r is the measured anisotropy, and r₀ is the intrinsic anisotropy. The intrinsic anisotropy, r₀, corresponds to the anisotropy value expected when the fluorescent probe experiences no rotation during the excited lifetime of its fluorophore. The measured polarization, P, of the fluorescent probe can be converted to anisotropy, r, using the expression

$$r = \frac{2 * P}{3 - P} \qquad ( 2 )$$

For a given fluorescent probe, the product of CᵣTτ is able to be approximated as a constant and can be experimentally calculated. For an illustrative MIP/microplate setup, the value of CᵣTτ was calculated to be 0.5 poise.

In order to achieve consistently accurate estimations of microviscosity using MIP and a microplate reader, it was found that the value of r₀ must be recalculated for each experiment. The value of r₀ can be recalculated by using the anisotropy value of MIP in water that is analyzed alongside the sample of interest to calibrate the system. This is done by rearranging Equation 1 to the following form

$${r}_{0} = \frac{{C}_{r} T τ {r}_{w}}{{η}_{w , cal}} + {r}_{w} \qquad ( 3 )$$

where r_{w} is the measured anisotropy of MIP in water after background subtraction and η_{w,cal} is the microviscosity estimate of water in units of poise to which the system will be calibrated. Background subtraction is performed by subtracting the average parallel and perpendicular intensities of a blank sample from the respective parallel and perpendicular intensities of the sample of interest. As the value of r_{w} varies slightly from experiment to experiment, the calculated value of r₀ thus does too. For 100 nm and 500 nm MIP, the calibration values of η_{w,cal} that are found to give the most accurate estimates of microviscosity are 30×10⁻² and 35×10⁻² poise respectively. This differs from the true viscosity of water of 8.9×10⁻⁴ poise and likely represents the limit of detection to measure microviscosity above 30×10⁻² poise (30 cP). Microviscosity is calculated by inserting the calculated r₀ from Equation 3, the calculated value of Cᵣtτ, and the measured r of the fluorescent probe in the sample of interest after background subtraction into Equation 1.

Correlation of Rotational Diffusion of MIP with Mucus Microrheology

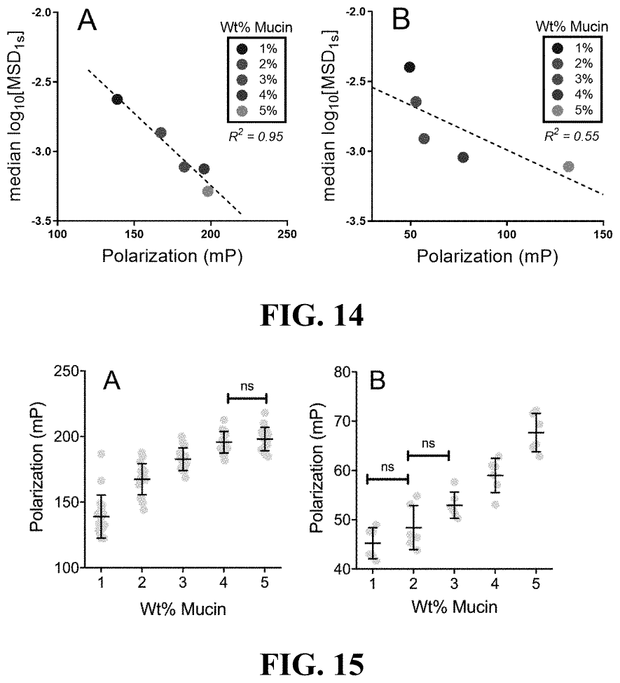

The rotational diffusion of 100 nm and 500 nm MIP was found to be strongly correlated with MIP translational diffusion in mucus hydrogels composed of 1-5% PGM and 2% PEG-4SH w/v. As the MIP translational diffusion decreased in successively higher concentration mucin hydrogels, the corresponding rotational diffusion also decreased, as represented by a higher polarization value (FIG. 14). Since the translational diffusion of MIP is a previously validated measure of microrheology, these results indicate that rotational diffusion of MIP also correlates with mucus microrheology.

Additionally, the rotational diffusion of both 100 nm and 500 nm MIP was found to be sensitive to increasing mucin concentration in a model mucus hydrogel (FIG. 15). The mucin concentrations used span the physiological range expected in healthy airways and in the airways of individuals with obstructive lung diseases. Interestingly, it was found that 100 nm MIP were insensitive to high concentrations (≥4 wt %), unlike 500 nm MIP which were determined to be most sensitive at higher wt% mucin. These data demonstrate the potential of this approach for monitoring disease progression in patients with chronic obstructive lung diseases.

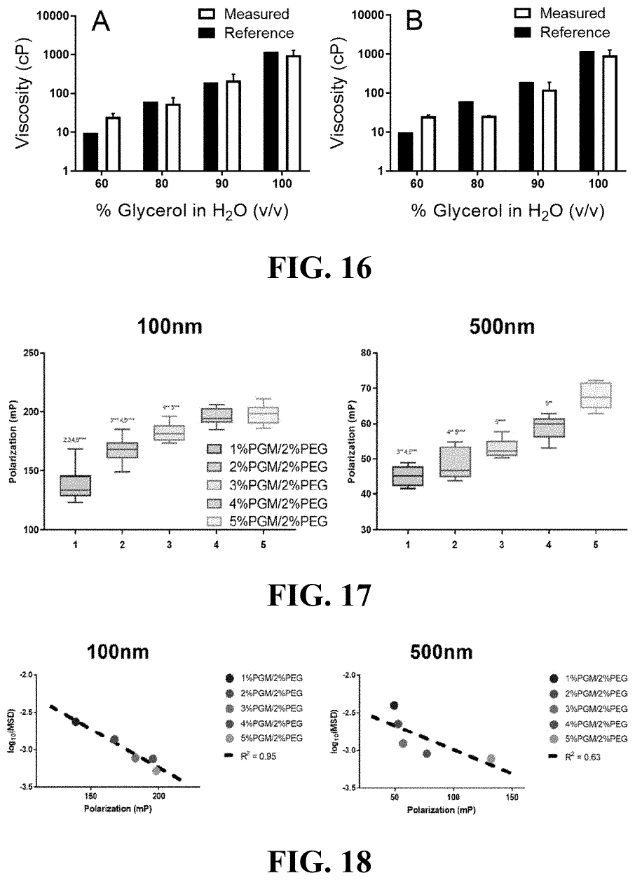

Further, the viscosities of 60-100% v/v glycerol solutions were able to be accurately estimated using the measured rotational diffusion of 100 nm and 500 nm MIP, as obtained through FP (FIG. 16). The ability to accurately estimate viscosity from rotational diffusion measurements enables physical interpretation of results, for determining the absence, or alternatively the presence, progression, and prognosis of obstructive lung diseases.

### EXAMPLE 3

Muco-inert particles (MIP) were made by attaching a dense layer of polyethylene glycol (PEG) to the surface of 100 nm and 500 nm polystyrene nanoparticles. Rotational diffusion of MIP was measured in a mucus hydrogel model using fluorescence polarization, with the results shown in FIG. 17.

FIG. 17 shows rotational diffusion results, for polarization values (Polarization (mP)) as a function of concentration, for rotational diffusion of 100 nm and 500 nm MIP in 1-5% porcine gastric mucin (PGM) with 2% 4-arm PEG-SH. Rotational diffusion of 100 nm and 500 nm MIP decreased with increasing mucin concentration. Polarization values were obtained through fluorescence polarization.

The results in FIG. 17 show that MIP rotational diffusion was reduced in a concentration—dependent manner in the mucus hydrogel model.

Rotational diffusion and log 10(MSD) determinations then were made with the results shown in FIG. 18 for rotational diffusion and log₁₀(MSD) of 100 nm and 500 nm MIP in 1-5% PGM with 2% 4-arm PEG-SH. Decrease in translational diffusion of 100 nm and 500 nm MIP was associated with decreased in corresponding rotational diffusion. MSD values were obtained through high-speed video microscopy and multiple particle tracking.

The results in FIG. 18 show that MIP rotational diffusion correlates with translational diffusion in the mucus samples.

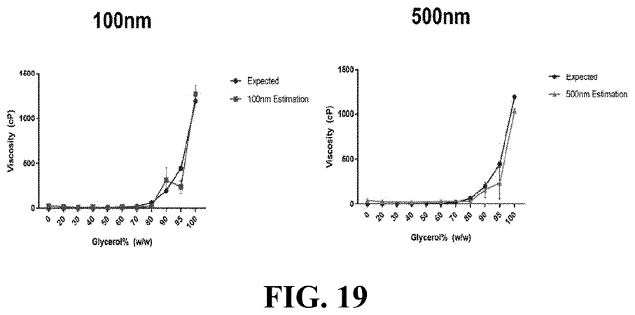

Estimations then were made against measured values for viscosity of aqueous glycerol solutions at varying glycerol concentrations, based on the 100 nm and 500 nm MIP rotational diffusion results, for which data are shown in FIG. 19.

As shown by such data, accurate estimations of the viscosity of glycerol solutions at various glycerol concentrations was obtained from both 100 nm and 500 nm MIP polarization values.

The foregoing results show that rotational diffusion of 100 nm and 500 nm MIP is (i) sensitive to mucin concentration, which is an established biomarker of obstructive lung diseases, (ii) correlated with translational diffusion of MIP, a validated measure of mucus microrheology, and (iii) capable of accurately estimating viscosity of solutions and hydrogels, using clinically-available equipment and technology.

While the disclosure has been set forth herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the disclosure as hereinafter claimed is intended to be broadly construed and interpreted, as including all such variations, modifications and alternative embodiments, within its spirit and scope.

### BIBLIOGRAPHY

1. Bansil, R.; Turner, B. S., The biology of mucus: Composition, synthesis and organization. Advanced drug delivery reviews 2018, 124, 3-15.

2. Linden, S.; Sutton, P.; Karlsson, N.; Korolik, V.; McGuckin, M., Mucins in the mucosal barrier to infection. Mucosal immunology 2008, 1 (3), 183.

3. Thornton, D. J.; Sheehan, J. K., From mucins to mucus: toward a more coherent understanding of this essential barrier. Proceedings of the American Thoracic Society 2004, 1 (1), 54-61.

4. Perez-Vilar, J.; Hill, R. L., The structure and assembly of secreted mucins. Journal of Biological Chemistry 1999, 274 (45), 31751-31754.

5. Lai, S. K.; Wang, Y.-Y.; Wirtz, D.; Hanes, J., Micro-and macrorheology of mucus. Advanced drug delivery reviews 2009, 61 (2), 86-100.

6. Cone, R. A., Barrier properties of mucus. Advanced drug delivery reviews 2009, 61 (2), 75-85.

7. Wagner, C. E.; Turner, B. S.; Rubinstein, M.; McKinley, G. H.; Ribbeck, K., A rheological study of the association and dynamics of MUC5AC gels. Biomacromolecules 2017, 18 (11), 3654-3664.

8. Werlang, C.; Cárcarmo-Oyarce, G.; Ribbeck, K., Engineering mucus to study and influence the microbiome. Nature Reviews Materials 2019, 1.

9. Carlson, T.; Lock, J.; Carrier, R., Engineering the mucus barrier. Annual review of biomedical engineering 2018, 20, 197-220.

10. Duncan, G. A.; Jung, J.; Hanes, J.; Suk, J. S., The mucus barrier to inhaled gene therapy. Molecular Therapy 2016, 24 (12), 2043-2053.

11. Berry, M.; Corfield, A. P.; McMaster, T. J., Mucins: a dynamic biology. Soft Matter 2013, 9 (6), 1740-1743.

12. Celli, J. P.; Turner, B. S.; Afdhal, N. H.; Ewoldt, R. H.; McKinley, G. H.; Bansil, R.; Erramilli, S., Rheology of gastric mucin exhibits a pH-dependent sol-gel transition. Biomacromolecules 2007, 8 (5), 1580-1586.

13. Kočevar-Nared, J.; Kristl, J.; Šmid-Korbar, J., Comparative rheological investigation of crude gastric mucin and natural gastric mucus. Biomaterials 1997, 18 (9), 677-681.

14. Hamed, R.; Fiegel, J., Synthetic tracheal mucus with native rheological and surface tension properties. Journal of biomedical materials research Part A 2014, 102 (6), 1788-1798.

15. Duncan, G. A.; Jung, J.; Joseph, A.; Thaxton, A. L.; West, N. E.; Boyle, M. P.; Hanes, J.; Suk, J. S., Microstructural alterations of sputum in cystic fibrosis lung disease. JCI Insight 2016, 1 (18), e88198.

16. Davies, J. R.; Carlstedt, I., Isolation of large gel-forming mucins. In Glycoprotein Methods and Protocols, Springer: 2000; pp 3-13.

17. Schömig, V. J.; Käsdorf, B. T.; Scholz, C.; Bidmon, K.; Lieleg, O.; Berensmeier, S., An optimized purification process for porcine gastric mucin with preservation of its native functional properties. RSC Advances 2016, 6 (50), 44932-44943.

18. Cao, X.; Bansil, R.; Bhaskar, K. R.; Turner, B. S.; LaMont, J. T.; Niu, N.; Afdhal, N. H., pH-dependent conformational change of gastric mucin leads to sol-gel transition. Biophysical journal 1999, 76 (3), 1250-1258.

19. Georgiades, P.; Pudney, P. D.; Thornton, D. J.; Waigh, T. A., Particle tracking microrheology of purified gastrointestinal mucins. Biopolymers 2014, 101 (4), 366-377.

20. Hill, D. B.; Vasquez, P. a.; Mellnik, J.; McKinley, S. a.; Vose, A.; Mu, F.; Henderson, A. G.; Donaldson, S. H.; Alexis, N. E.; Boucher, R. C.; Forest, M. G., A biophysical basis for mucus solids concentration as a candidate biomarker for airways disease. PLoS ONE 2014, 9, 1-11.

21. Button, B.; Goodell, H. P.; Atieh, E.; Chen, Y.-C.; Williams, R.; Shenoy, S.; Lackey, E.; Shenkute, N. T.; Cai, L.-H.; Dennis, R. G., Roles of mucus adhesion and cohesion in cough clearance. Proceedings of the National Academy of Sciences 2018, 115 (49), 12501-12506.

22. Schuster, B. S.; Suk, J. S.; Woodworth, G. F.; Hanes, J., Nanoparticle diffusion in respiratory mucus from humans without lung disease. Biomaterials 2013, 34 (13), 3439-46.

23. Sechi, S.; Chait, B. T., Modification of cysteine residues by alkylation. A tool in peptide mapping and protein identification. Analytical Chemistry 1998, 70 (24), 5150-5158.

24. Suk, J. S.; Lai, S. K.; Boylan, N. J.; Dawson, M. R.; Boyle, M. P.; Hanes, J., Rapid transport of muco-inert nanoparticles in cystic fibrosis sputum treated with N-acetyl cysteine. Nanomedicine 2011, 6 (2), 365-375.

25. Yuan, S.; Hollinger, M.; Lachowicz-Scroggins, M. E.; Kerr, S. C.; Dunican, E. M.; Daniel, B. M.; Ghosh, S.; Erzurum, S. C.; Willard, B.; Hazen, S. L.; Huang, X.; Carrington, S. D.; Oscarson, S.; Fahy, J. V., Oxidation increases mucin polymer cross-links to stiffen airway mucus gels. Science translational medicine 2015, 7, 276ra27.

26. Schuster, B. S.; Ensign, L. M.; Allan, D. B.; Suk, J. S.; Hanes, J., Particle tracking in drug and gene delivery research: State-of-the-art applications and methods. Adv Drug Deliv Rev 2015, 91, 70-91.

27. Mason, T. G.; Weitz, D., Optical measurements of frequency-dependent linear viscoelastic moduli of complex fluids. Physical review letters 1995, 74 (7), 1250.

28. Shin, J. H.; Gardel, M.; Mahadevan, L.; Matsudaira, P.; Weitz, D., Relating microstructure to rheology of a bundled and cross-linked F-actin network in vitro. Proceedings of the National Academy of Sciences 2004, 101 (26), 9636-9641.

29. De Gennes, P.-G.; Gennes, P.-G., Scaling concepts in polymer physics. Cornell university press: 1979.

30. Savin, T.; Doyle, P. S., Electrostatically tuned rate of peptide self-assembly resolved by multiple particle tracking. Soft Matter 2007, 3 (9), 1194-1202.

31. Dawson, M.; Wirtz, D.; Hanes, J , Enhanced viscoelasticity of human cystic fibrotic sputum correlates with increasing microheterogeneity in particle transport. The Journal of biological chemistry 2003, 278, 50393-401.

32. Lai, S. K.; Wang, Y. Y.; Hida, K.; Cone, R.; Hanes, J., Nanoparticles reveal that human cervicovaginal mucus is riddled with pores larger than viruses. Proc Natl Acad Sci USA 2010, 107 (2), 598-603.

33. Valentine, M. T.; Kaplan, P. D.; Thota, D.; Crocker, J. C.; Gisler, T.; Prud'homme, R. K.; Beck, M.; Weitz, D. A., Investigating the microenvironments of inhomogeneous soft materials with multiple particle tracking. Physical Review E 2001, 64 (6), 061506.

34. Cicuta, P.; Donald, A. M., Microrheology: a review of the method and applications. Soft Matter 2007, 3 (12), 1449-1455.

35. Xu, Q.; Ensign, L. M.; Boylan, N. J.; Schon, A.; Gong, X.; Yang, J.-C.; Lamb, N. W.; Cai, S.; Yu, T.; Freire, E., Impact of surface polyethylene glycol (PEG) density on biodegradable nanoparticle transport in mucus ex vivo and distribution in vivo. ACS nano 2015, 9 (9), 9217-9227.

36. Ensign, L. M.; Schneider, C.; Suk, J. S.; Cone, R.; Hanes, J., Mucus penetrating nanoparticles: biophysical tool and method of drug and gene delivery. Advanced Materials 2012, 24 (28), 3887-3894.

37. Huckaby, J. T.; Lai, S. K., PEGylation for enhancing nanoparticle diffusion in mucus. Advanced drug delivery reviews 2018, 124, 125-139.

38. Mason, T.; Ganesan, K.; Van Zanten, J.; Wirtz, D.; Kuo, S., Particle tracking microrheology of complex fluids. Physical review letters 1997, 79 (17), 3282.

39. Thornton, D. J.; Khan, N.; Mehrotra, R.; Howard, M.; Sheehan, J. K.; Veerman, E.; Packer, N. H., Salivary mucin MG1 is comprised almost entirely of different glycosylated forms of the MUCSB gene product. Glycobiology 1999, 9 (3), 293-302.

40. Suk, J. S.; Lai, S. K.; Wang, Y. Y.; Ensign, L. M.; Zeitlin, P. L.; Boyle, M. P.; Hanes, J., The penetration of fresh undiluted sputum expectorated by cystic fibrosis patients by non-adhesive polymer nanoparticles. Biomaterials 2009, 30 (13), 2591-7.

41. Lai, S. K.; O'Hanlon, D. E.; Harrold, S.; Man, S. T.; Wang, Y. Y.; Cone, R.; Hanes, J., Rapid transport of large polymeric nanoparticles in fresh undiluted human mucus. Proc Natl Acad Sci USA 2007, 104 (5), 1482-7.

42. Fahy, J. V. & Dickey, B. F. Airway mucus function and dysfunction. The New England journal of medicine 2010, 363, 2233-2247, doi:10.1056/NEJMra0910061.

43. Shinitzky, M. & Barenholz, Y. Fluidity parameters of lipid regions determined by fluorescence polarization. Biochimica et Biophysica Acta (BBA)—Reviews on Biomembranes 1978, 515, 367-394, doi:https://doi.org/10.1016/0304-4157(78)90010-2.

What is claimed is:

1. A synthetic hydrogel, comprising hydrated mucin glycoproteins cross-linked with multi-aim thiol functional cross-linker.

2. The synthetic hydrogel of claim 1, wherein the multi-arm thiol functional cross-linker comprises thiol functionality at the termini of multiple ones of its arms.

3. The synthetic hydrogel of claim 1, wherein the multi-arm thiol functional cross-linker has four arms, each of which is linked at a central organic core structure and extends outwardly therefrom, and comprises polyalkyloxy linear segments and a terminal thiol functionality.

4. The synthetic hydrogel of claim 1, wherein each arm of the multi-armor thiol functional cross-linker comprises a chain structure of the formula —O(CH₂CH₂O)ₙCH₂CH₂SH wherein n is in a range of from 1 to 1000.

5. The synthetic hydrogel of claim 1, wherein the mucin glycoproteins are porcine mucin glycoproteins, bovine mucin glycoproteins, or human mucin glycoproteins.

6. The synthetic hydrogel of claim 1, wherein the multi-arm thiol functional cross-linker comprises a 4-arm polyethylene glycol thiol of the structure

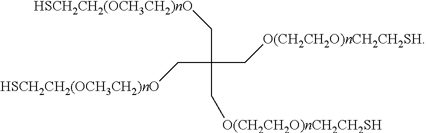

7. The synthetic hydrogel of claim 6, wherein the mucin glycoproteins are porcine mucin glycoproteins or bovine mucin glycoproteins.

8. The synthetic hydrogel of claim 1, having an elastic modulus G′ of from 100 to 400 Pa in an angular frequency range of 0.1 to 200 rad/sec, and a viscous modulus G″ of from 3 to 90 Pa in an angular frequency range of 0.1 to 200 rad/sec, when measured at pH 7.4 and 37° C.

9. The synthetic hydrogel of claim 1, wherein:
the multi-arm thiol functional cross-linker comprises a 4-arm polyethylene glycol thiol of the structure 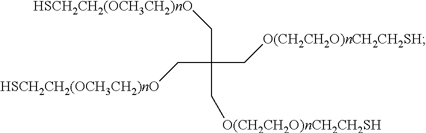
the mucin glycoproteins are porcine mucin glycoproteins or bovine mucin glycoproteins; and
the synthetic hydrogel has an elastic modulus G′ of from 100 to 400 Pa in an angular frequency range of 0.1 to 200 rad/sec, and a viscous modulus G″ of from 3 to 90 Pa in an angular frequency range of 0.1 to 200 rad/sec, when measured at pH 7.4 and 37° C.

10. A method of making a synthetic hydrogel, comprising:
combining mucin in aqueous medium with a multi-arm thiol functional cross-linker; and
cross-linking the mucin with the multi-arm thiol functional cross-linker to form the synthetic hydrogel.

11. The method of claim 10, wherein the aqueous medium is a buffered aqueous medium.

12. The method of claim 10, wherein the mucin comprises porcine mucin or bovine mucin, in dry powder form.

13. The method of claim 10, wherein the method is conducted to form the synthetic hydrogel, having an elastic modulus G′ of from 100 to 400 Pa in an angular frequency range of 0.1 to 200 rad/sec, and a viscous modulus G″ of from 3 to 90 Pa in an angular frequency range of 0.1 to 200 rad/sec, when measured at pH 7.4 and 37° C.

14. The method of claim 10, wherein the multi-arm thiol functional cross-linker comprises a 4-aim polyethylene glycol thiol of the structure

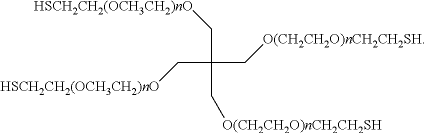

15. A method of microrheologically characterizing mucus, comprising:
dispersing in the mucus muco-inert particles (MIP);
irradiating the mucus containing MIP with polarized light; and
measuring fluorescence polarization (FP) resulting from rotational diffusion of the MIP in the mucus in response to said irradiating, as a microrheological characteristic of the mucus.

16. The method of claim 15, wherein the mucus containing MIP is formed in or introduced to a well of a plate, and said FP is measured using a plate reader equipped with a spectrofluorometer and polarized filter set, to which the plate having the mucus containing MIP in the well thereof is introduced for the measuring.

17. The method of claim 15, further comprising determining microviscosity of the mucus based on the measured FP.

18. The method of claim 15, wherein the MIP have a size in a range of from 50 nm to 1000 nm.

19. The method of claim 15, wherein the MIP comprise polymeric nanoparticles that are coated with a mucus adhesion-resistant coating.

20. The method of claim 15, wherein the mucus is microrheologically characterized to identify presence, absence, progression, or prognosis of obstructive lung disease.
